(19) United States
(12) Patent Application Publication
(10) Pub. No.: US 2004/0243019 A1
(43) Pub. Date: Dec. 2, 2004

(54) Weighted gradient method and system for diagnosing disease

(75) Inventors: Milan Graovac, Toronto (CA); Zoran Pavlovic, Toronto (CA)

Correspondence Address: BERESKIN AND PARR, SCOTIA PLAZA, 40 KING STREET WEST-SUITE 4000 BOX 401, TORONTO, ON, M5H 3Y2, CA

(73) Assignee: Z-Tech (Canada) Inc., Toronto (CA)

(21) Appl. No.: 10/806,186

(22) Filed: Mar. 23, 2004

Related U.S. Application Data
(63) Continuation-in-part of application No. 10/397,331, filed on Mar. 27, 2003, now abandoned.

(51) Int. Cl. A61B 5/05

(52) U.S. Cl. 600/547

(57) ABSTRACT

A method for detecting and diagnosing disease states in a body part is described. The method starts with a preparatory step of modeling the body part as a grid of many finite elements, then calculating the effect of the electrical property of each finite element at any one of a plurality of electrodes on the periphery of the body part as a function of the position of the finite element within the grid. This is termed the weight (influence) of the element. With this baseline information, electrical impedance measurements made at the plurality of electrodes on the periphery of the body part can be used in a diagnostic module to calculate a Weighted Element Value (WEVal) for each element. In a preferred embodiment of invention, the difference in WEVal magnitude between corresponding elements of homologous body parts serves as an indicator of the presence of disease.

### CROSS REFERENCES TO RELATED APPLICATIONS

This application is a continuation-in-part of Application No. 10/397,331, filed on Mar. 27, 2003, the contents of which are incorporated herein by reference.

### FIELD OF THE INVENTION

This invention relates to a method for detecting and diagnosing disease states in living organisms and specifically relates to diagnosis of disease by measuring electrical properties of body parts.

### BACKGROUND OF THE INVENTION

Several methods exist for diagnosing disease that involve measuring a physical property of a part of the body. A change in such a physical property can signal the presence of disease. For example, x-ray techniques measure tissue physical density, ultrasound measures acoustic density, and thermal sensing techniques measures differences in tissue heat generation and conduction. Other properties are electrical, such as the impedance of a body part that is related to the resistance that the body part offers to the flow of electrical current through it.

Values of electrical impedance of various body tissues are well known through studies on intact humans or from excised tissue made available following therapeutic surgical procedures. In addition, it is well documented that a decrease in electrical impedance occurs in tissue as it undergoes cancerous changes. This finding is consistent over many animal species and tissue types, including, for example human breast cancers.

There have been a number of reports of attempts to detect breast tumors using electrical impedance imaging, such as, for example, U.S. Pat. No. 4,486,835. However, there are basic problems when trying to construct an image from impedance data. Electric current does not proceed in straight lines or in a single plane; it follows the path of least resistance, which is inevitably irregular and three-dimensional. As a result, the mathematics for constructing the impedance is very complex and requires simplifying assumptions that greatly decrease image fidelity and resolution.

Despite such difficulties, a method that permits comparisons of electrical properties for diagnostic purposes has been developed that involves homologous body parts, i.e., body parts that are substantially similar, such as a left breast and a right breast. In this method, the impedance of a body part of a patient is compared to the impedance of the homologous body part of the same patient. One technique for screening and diagnosing diseased states within the body using electrical impedance is disclosed in U.S. Pat. No. 6,122,544, which is incorporated herein by reference. In this patent, data are obtained from two anatomically homologous body regions, one of which may be affected by disease. Differences in the electrical properties of the two homologous body parts could signal disease. One subset of the data so obtained is processed and analyzed by structuring the data values as elements of an n×n impedance matrix. The matrices can be further characterized by their eigenvalues and eigenvectors. These matrices and/or their eigenvalues and eigenvectors can be subjected to a pattern recognition process to match for known normal or disease matrix or eigenvalue and eigenvectors patterns. The matrices and/or their eigenvalues and eigenvectors derived from each homologous body region can also be compared, respectively, to each other using various analytical methods and then subjected to criteria established for differentiating normal from diseased states.

Published international patent application, PCT/CA01/01788, which is incorporated herein by reference, discloses a breast electrode array for diagnosing the presence of a disease state in a living organism, wherein the electrode array comprises a flexible body, a plurality of flexible arms extending from the body, and a plurality of electrodes provided by the plurality of flexible arms, wherein the electrodes are arranged on the arms to obtain impedance measurements between respective electrodes. In one embodiment, the plurality of flexible arms are spaced around the flexible body and are provided with an electrode pair. In operation, the electrodes are selected so that the impedance data obtained will include elements of an n×n impedance matrix, plus other impedance values that are typically obtained with tetrapolar impedance measurements. Tetrapolar impedance measurements are associated with injecting current between so called current electrodes and measuring a voltage drop between associated electrodes. In a preferred embodiment, the differences between corresponding homologous impedance measurements in the two body parts are compared in a variety of ways that allow the calculation of metrics that can serve to either indicate the presence of disease or localize the disease to a specific breast quadrant or sector. The impedance differences are also displayed graphically, for example in a frontal plane representation of the breast by partitioning the impedance differences into pixel elements throughout the plane.

Despite the attractive features of this method of diagnosing disease in one of a homologous pair of body parts, there are some problems associated with this straightforward implementation. In particular, the current path through the body part, whether healthy or not, as the current flows from one electrode to the other is, in general, complex. It encompasses to a certain extent, all areas of the body part. In the aforementioned method, this complexity is addressed by simplifying assumptions. This simplification may affect the ability of the method to detect the disease.

### SUMMARY OF THE INVENTION

The present invention is directed to an improved method for detecting and diagnosing disease states in a living organism by using a set of electrical impedance measurements. The method is based on the realistic distribution of electric current in the body part. For each impedance measurement, the approximate current distribution is obtained by a numerical computation using a representation of a body part structure, or by the direct measurement performed on a physical model or a control subject's body part. This obtained current distribution is further used to correlate impedances obtained by direct measurements to different areas in the body part.

To achieve this goal, the subject body part is subdivided into a number of small regions called finite elements. For each of the elements and for each of the electrode pairs used to inject current into the body part, a weight factor (obtained by computing or measuring the current density in the element), reflecting the position of the element within the body part, is calculated and stored. Each element has one weight factor for each current injection. Larger weight factors are associated with current injections that result in larger current densities in a particular element. Thus, current injecting scenarios associated with larger weights at a particular element are given greater consideration when detecting disease. The weights are typically calculated or measured with the assumption that there is no disease present. At the same time, baseline impedances associated with each of the current injections are obtained. The weights and baseline impedances for each of the current injection scenarios are stored in the database and used when a diagnosis is made following the measurement of the actual impedances of the subject's body part. For each element, the diagnostic is the sum over all current injections of weight multiplied by the ratio of baseline to measured impedance. This sum is referred to as a Weighted Element Value (WEVal). The higher the value of the sum is, the higher is the probability of the disease at the location of a particular element. Elements are grouped according to known physical characteristics and a sum for each of the groups is obtained. Comparing sums of homologous regions may point to a presence of disease in the body part.

In particular, a system and method for diagnosing the possibility of disease in a body part is described herein. The system includes an electrode array by which an electrical property of the body part may be measured, such as a measured impedance. The system further includes a grid module for representing the body part with a grid having a plurality of finite elements, and for obtaining a baseline electrical property using a model of the body part, such as a baseline impedance. The system also includes a weight module for using the model of the body part to compute a set of weights associated with a particular one of the plurality of finite elements, each weight in the set derived from a particular current injection electrode pair selection. A diagnostic module computes a diagnostic at the particular finite element to diagnose the possibility of disease in the body part, the diagnostic being a function of the measured electrical property, the baseline electrical property and the set of weights.

### BRIEF DESCRIPTION OF THE DRAWINGS

FIG. 1A shows the components of a basic tetrapolar measurement;

FIG. 1B is a block diagram of a system for detecting and diagnosing disease in a body part;

FIG. 1C is a block data flow diagram of a method for detecting and diagnosing disease in a body part;

FIG. 2 is a sample finite element grid produced by the grid module of FIG. 1B, the grid representing a body part that can be used to calculate baseline electrical properties;

FIG. 3 is a block data flow diagram of the grid module of FIG. 1B, in one embodiment of the present invention that employs a numerical finite element method;

FIG. 4 is a block data flow diagram of the diagnostic module of FIG. 1B, in one embodiment of the present invention;

FIG. 5 is a flowchart illustrating the method steps performed by the diagnostic system of FIG. 1B to diagnose disease; and

FIGS. 6A and 6B are sample WEVal plots for an actual subject that can be used to detect breast cancer.

### DETAILED DESCRIPTION OF THE INVENTION

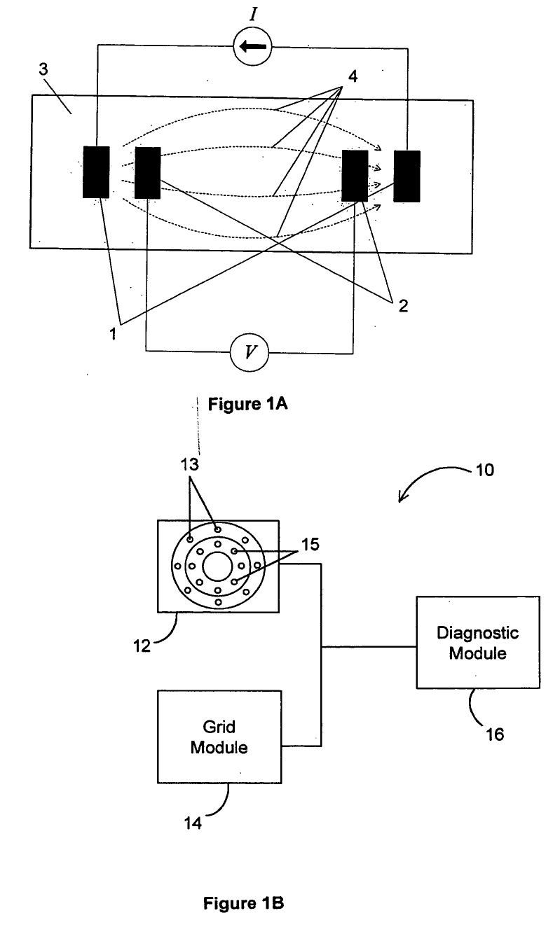

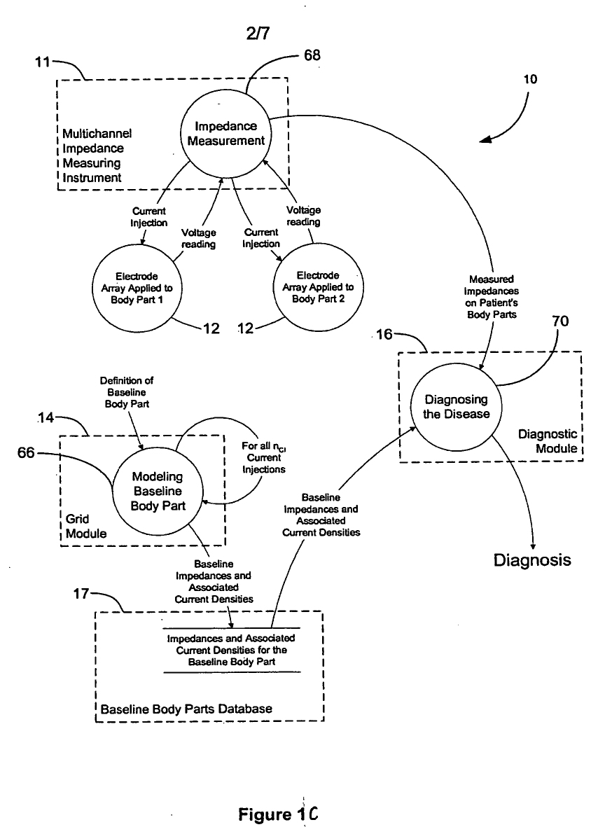

FIG. 1A shows a schematic of components used to perform a tetrapolar impedance measurement, which measurements are used for detecting and diagnosing disease, as described in more detail below. FIGS. 1B and 1C show a block diagram of a system 10 and an outline of a method for detecting and diagnosing disease in a body part, such as breast cancer. The method uses impedance measurements taken from a multi-channel impedance measuring instrument 11 with a pair of electrode arrays 12, like the one described in PCT/CA01/01788, a grid module 14 and a diagnostic module 16.

Referring to FIG. 1A, a single electrical impedance measurement is performed using four electrodes. One pair of electrodes 1 is used for the application of current I, and the other pair of electrodes 2 is used to measure the voltage V that is produced across a material, such as breast tissue 3, by the current. The current I flowing between electrodes 1 is indicated by the arrows 4. The impedance Z is the ratio of V to I; i.e., Z=V/I. By using separate electrode pairs for current injection and voltage measurement, polarization effects at the voltage measurement electrodes 2 are minimized and a more accurate measurement of impedance can be produced. It should be understood that, in general, the voltage electrodes 2 need not be disposed between the two current electrodes 1.

Impedance consists of two components, resistance and capacitive reactance (or equivalently, the magnitude of impedance and its phase angle). Both components are measured and analyzed in the present invention. However, in examples described below, only resistance is used and interchangeably referred to as either resistance or the more general term impedance.

As has been noted above, by performing tetrapolar measurements in which separate electrode pairs are used for current injection and voltage measurement, polarization effects at the voltage measurement electrodes 2 are minimized and more accurate measurements of impedance can be performed. However, there may be some embodiments in which bipolar, instead of a tetrapolar, measurements can be performed as part of the general method for diagnosing disease discussed below. If bipolar measurements are performed, a correction factor can be used that corrects for the polarization effects arising from skin-to-electrode interface.

FIG. 1B shows a schematic of the electrode array 12. Eight current injection electrodes 13, and eight associated voltage measurement electrodes 15 are shown. In general, there are nₑ current injection electrodes and nₑ associated voltage measurement electrodes in the electrode array. The electrodes are applied on the body part, each of the current injection electrodes being associated with the adjacent voltage measurement electrode. Impedance is measured between two voltage electrodes when the current is injected between associated current electrodes. Since there are n_{CI}=nₑ·(nₑ−1)/2 pairs of current injection electrodes, and an equal number of voltage measurement electrode pairs, the total number of independent current injections and related impedances is n_{CI}. It should be understood that the electrode array shown is but one possible electrode array. Other electrode arrays may also be used.

As discussed in more detail below, the grid module 14 uses a numerical or physical model of a baseline (idealized or reference) body part to compute baseline values. In particular, at step (66), baseline impedances and associated gradients for the baseline body part are calculated in the grid module 14. As detailed below, the associated gradients can be used to calculate current densities at each finite element. The baseline impedances for each of the n_{CI} current injections, and the associated current densities for each of the finite elements and for each of the n_{CI} current injections are stored in a baseline body parts database 17.

At step (68), the impedance is measured n_{CI} times resulting in the set of values, {Z₁^{M}, Z₂^{M}, . . . , Zₙ_{_{CI}}^{M}}, where Zⱼ^{M} is the impedance measured between the voltage electrodes associated with the j^{th} current injection electrode pair when current is injected between that current injection electrode pair, as required in tetrapolar impedance measurement.

The grid module 14 includes software and/or hardware for representing the body part with a grid of elements that are so small that the voltage gradient during arbitrary current injection is approximately constant within any single element. For example, if the body part is modeled as a two-dimensional surface, then the grid can be composed of triangles that “tile” the surface. Alternatively, the body part can be modeled by a three-dimensional grid whose elements are tetrahedrons, for example. Each finite element is associated with a plurality of nodes, typically on the perimeter of the finite element. As well, each finite element is characterized by its electrical material property, namely resistivity and/or permittivity. Adjacent elements share the nodes associated with the common side or face. When the elements are small enough to ensure that the current density throughout the element is constant for each of the current injections, the voltage gradient throughout the element is also constant and proportional to the current density.

The grid module 14 also includes software and/or hardware for deriving the current density for each of the elements in the grid. It does this by calculating the current density using a numerical or physical model, or by using population study information, as discussed in more detail below.

The diagnostic module 16 includes software and/or hardware for detecting the presence of a tumor in the body part at step (70). As described in more detail below, the diagnosis is based on a diagnostic that is a function of the impedance measurements obtained from a subject using the impedance measuring instrument 11, and a weighting factor derived from the estimated value of the current density throughout the body part, obtained using grid module 14.

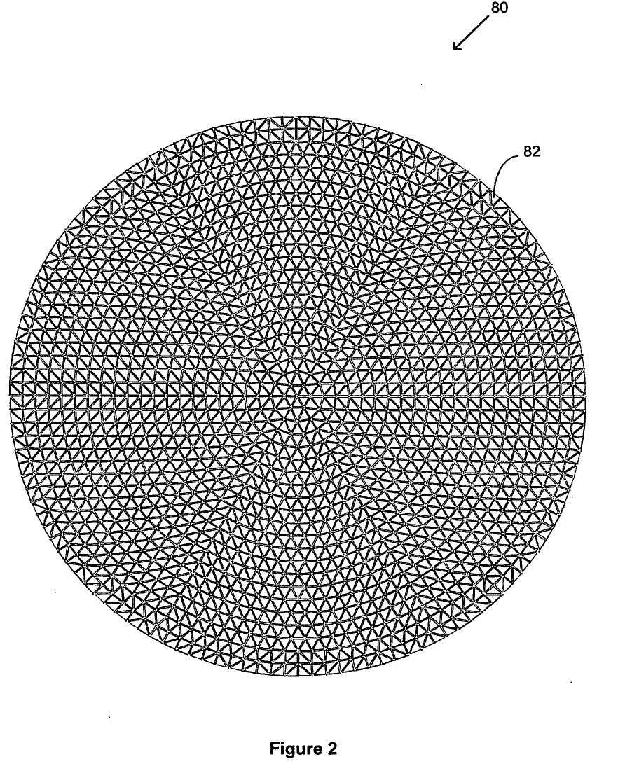

FIG. 2 shows a representation of the baseline body part divided into a grid 80 composed of a plurality of finite elements 82. Once the body part is subdivided using grid module 14 into a number of finite elements 82, there are several methods that can be used to calculate baseline values, such as the current density associated with a particular current injection and with a particular finite element 82 of the grid 80. FIG. 2 shows one embodiment of the present invention in which several thousand finite elements 82 are used, as required to justify linearizing the equations used to numerically compute the relevant electrical properties.

The preferred method used by the grid module 14 to associate a voltage gradient with a particular finite element 82 is a numerical finite element method that assumes that the resistivity of the body part is uniform. The method numerically solves Laplace's equation, known to those of ordinary skill, to compute the electric potential at the nodes of the finite element grid from which the electric voltage gradient can be obtained. Due to uniform resistivity, current density is proportional to the voltage gradient everywhere in the body part.

A second method that can be used by the grid module 14 is related to the last method, except that instead of assuming a uniform resistivity, more realistic resistivities and/or permittivities can be used that reflect the known internal structure of the body part. In this case the current density is proportional to the electric voltage gradient in each of the elements, but the voltage gradient to current density ratio depends on the resistivity and/or reactivity associated with the particular finite element 82.

The third method involves using a physical model of a typical breast. This typical breast acts as a baseline representation of the body part. The model is designed so that the measured impedance matrix is close to the average impedance matrix for the normal subject with the body part of the particular size. Each finite element 82 obtained using the grid module 14 is associated with the particular location (x, y and z coordinates) in the physical model. The current density at each of the finite elements 82 and for each of the current injections is obtained using one of the available instruments for measuring the current density. The current density instrument, for example, can be combined with magnetic resonance imaging (MRI) to measure and display the current density superimposed on the MRI image at any location of the body part model.

The fourth method is similar to the third method except that the measurement of the current density for each current injection and at the location of each of the finite elements 82 defined by the grid module 14 is performed on the body part of an actual control subject. For example, the same combination of instruments as above can be used to measure and display the current density superimposed on the MRI image at any location in the actual body part.

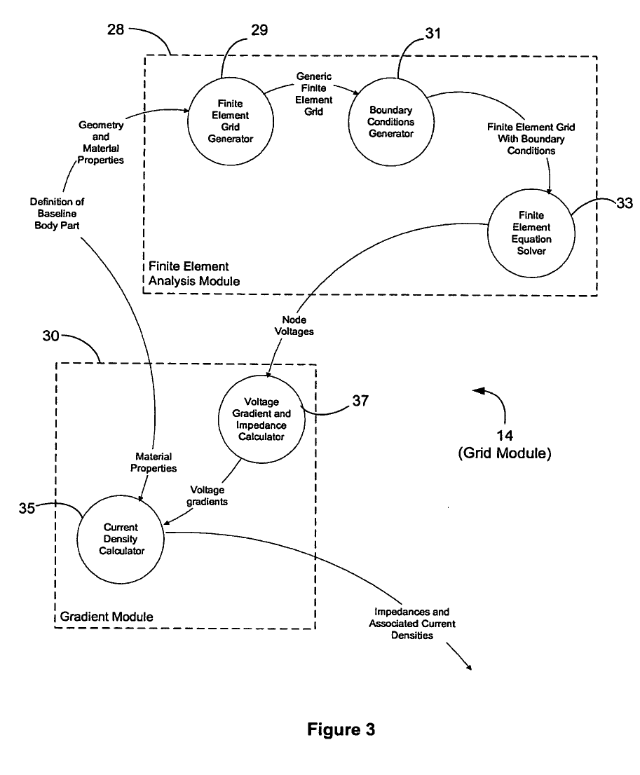

FIG. 3 shows a block data flow diagram of the grid module 14 in the preferred embodiment of the invention where it includes a finite element analysis module 28 and a gradient module 30.

In the preferred embodiment of the invention, for any single current injection, a finite element method is used to estimate baseline values for electric potential gradients and resulting current densities in each of the elements. In addition, the grid module 14 uses the finite element method to compute the baseline impedance. More generally, the baseline impedance refers to the impedance calculated by the grid module 14 (denoted by Zⱼ, for the j^{th} electrode pair) using an appropriate physical or numerical model, as distinguished from the measured impedance, Zⱼ^{M}, obtained by a measurement on a subject using an electrode array.

The finite element analysis module 28 includes hardware and/or software that employs various boundary conditions, corresponding to the injections of current between the various pairs of current injection electrodes 13 (FIG. 1B), to compute the electric potential at all the nodes in the grid. The node voltage Vⱼᵢ is the voltage that arises at the node j when a current injection i is applied, where the i^{th} current injection refers to the injection of current between the i^{th} current injection electrode pair.

Specifically, the finite element analysis module 28 includes a finite element grid generator 29, a boundary conditions generator 31 and a finite element equation solver 33. The finite element grid generator 29 generates a grid 80 of finite elements 82 that spans a representation of the body part. Position on the representation of the body part can be discretized if each finite element is associated with several nodes, typically on the perimeter of the finite element.

To compute the potential, V, as a function of position on the grid, Laplace's equation ∇²V=0 is solved using a numerical finite element method. The boundary conditions generator 31 assigns boundary conditions corresponding to the various n_{CI} current injections. The finite element equation solver 33 employs the numerical finite element method for solving Laplace's equation. Many different types of such methods can be used, such as a Lax differencing scheme for solving partial differential equations. Several other techniques known to those of ordinary skill in the art can be utilized.

In addition to finding the electric potential as a function of node position, the grid module 14 also finds voltage differences between voltage measurement electrodes 15. In particular, using boundary conditions corresponding to the current injected by the first pair of current injection electrodes yields V₁, the voltage drops between the first pair of voltage measurement electrodes. Using boundary conditions corresponding to the current injected by the second pair of current injection electrodes yields V₂, the voltage drop between the second pair of voltage measurement electrodes. Continuing in this manner yields all n_{CI} voltages {V₁, V₂, . . . , Vₙ_{_{CI}}}. Each time Laplace's equation is solved, the finite element method yields the potential at every node of the grid as well. The node voltage Vⱼᵢ is the voltage that arises at the node j when a current injection i is applied. The gradient module 30 utilizes the calculated node voltages to find an estimated current density at the element k for the current injection i, Jᵢₖ. The grid module 14 similarly obtains all n_{CI} impedances {Z₁, Z₂, . . . , Zₙ_{_{CI}}} and all the current densities {J₁ₖ, J₂ₖ, . . . , Jₙ_{_{CI}}^{k}}, at the finite element k. In particular, to obtain Jᵢₖ, where Jᵢₖ is the magnitude of the current density in the k^{th} finite element for the current injection i, the gradient module 30 uses the electric potential at each node associated with finite element k. To this end, the magnitude of the gradient of the electric potential, which is equal to the magnitude of the electric field, is first obtained by a voltage gradient calculator 37.

For example, supposing the element to be two dimensional with potential V=φ(x,y), then E=|∇φ| where E is the magnitude of the electric field. The voltage gradient calculator 37 can obtain E as follows. In the (x,y,V) coordinate system, if θ is the angle between \hat{k}, the unit normal in the V direction, and the perpendicular to the surface V=φ(x,y), then tanθ=|∇φ|. To see this, an auxiliary function F(x,y,V)=V−φ(x,y) can be introduced. The quantity ∇F|∇F| is a normal vector perpendicular to the level surface F(x,y,V)=const., or, with const.=0, a normal vector perpendicular to the surface V=φ(x,y). Then,

$$\begin{matrix}\frac{sin θ}{cos θ} = \frac{\vert \hat{k} \times \frac{∇ V}{\vert ∇ V \vert} \vert}{\hat{k} \cdot \frac{∇ V}{\vert ∇ V \vert}} \\ = {[ {( \frac{\partial φ}{\partial x} )}^{2} + {( \frac{\partial φ}{\partial y} )}^{2} ]}^{1 / 2} \\ = \vert ∇ φ \vert \\ = E\end{matrix}$$

When employing the finite element analysis, the finite element analysis module 28 can either assume the body part to have a uniform resistance and/or reactance, or the resistance and/or reactance can be taken to be non-uniform to reflect the known structure of the body part.

A current density calculator 35 calculates the magnitude of the current density J from the magnitude of the electric field E and the tissue resistivity ρ using the microscopic version of Ohm's Law stating that at every point, J=E|ρ.

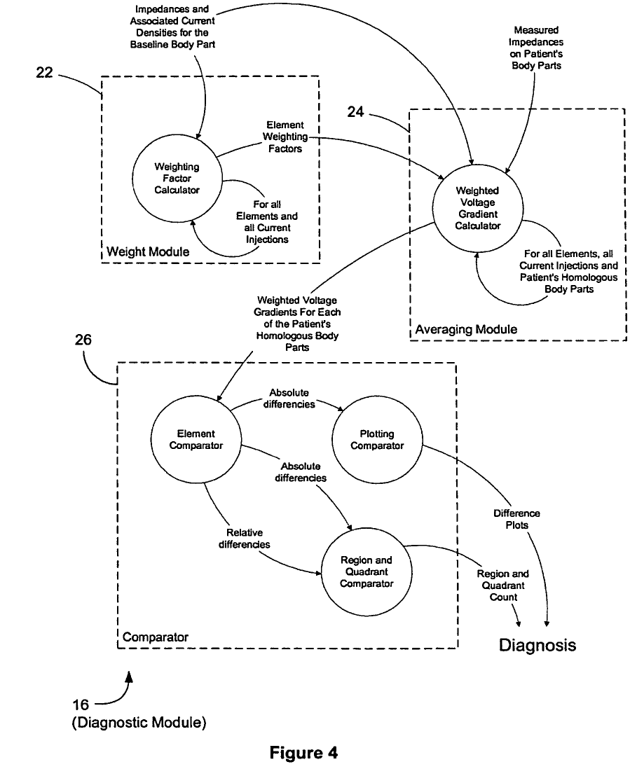

FIG. 4 shows a block data flow diagram of the diagnostic module 16 of FIG. 1B, in one embodiment of the present invention. The diagnostic module 16 includes a weight module 22, an averaging module 24 and a comparator 26.

As discussed previously, the diagnostic module 16 computes a Weighted Element Value (WEVal) parameter (diagnostic) at each of the finite elements 82 of the grid 80 representing the body part, and utilizes the diagnostic to diagnose the possibility of disease in the body part. The diagnostic is a function of the impedances and current densities calculated and/or measured for the baseline body part and impedances measured on the body part of the subject.

The weight module 22 includes software and/or hardware for calculating weights for the element k and the current injection i, wᵢₖ, given by

$${w}_{ik} = \frac{{J}_{ik}}{{\sum }_{j = 1}^{{n}_{CI}} {J}_{jk}} .$$

The quantity J₁ₖ is the magnitude of the current density, which exists at the finite element k when the reference current is applied between the first pair of current injection electrodes. The quantity J₂ₖ is the magnitude of the current density, which exists at the finite element k when the reference current is applied between the second pair of current injection electrodes, and so on.

The averaging module 24 includes software and/or hardware for calculating a weighted average of a function ƒ(Zᵢ,Zᵢ^{M}). The diagnostic at the finite element k is defined to be

$$⟨ {f}_{k} ⟩ = {\sum }_{i = 1}^{{n}_{CI}} {w}_{ik} f ( {Z}_{i} , {Z}_{i}^{M} ) .$$

The diagnostic [symbol] ƒₖ [symbol] is referred to as the Weighted Element Value (WEVal). The quantity Z₁ is the impedance between the first pair of electrodes for the baseline body part. The quantity Z₂ is the impedance between the second pair of electrodes for the baseline body part, and so on. The Zᵢ can be obtained using a numerical calculation or using a physical model (an artificial reproduction or the real body part of a control subject). The Zᵢ^{M} are obtained by direct measurement on the body part of a subject using an electrode array. In the preferred embodiment of the present invention, the function ƒ(Zᵢ,Zᵢ^{M}) is

$$f ( {Z}_{i} , {Z}_{i}^{M} ) = \frac{{Z}_{i}}{{Z}_{i}^{M}} .$$

It should be understood that other functions ƒ might be used in other embodiments, including functions that are independent of the baseline values Zᵢ. It should be further understood that the diagnostic module 16 can condition the raw measurements Zᵢ, such as by standardizing with a factor, etc, to find the diagnostic. Thus, in one embodiment, the function can be given by

$$f ( {Z}_{i} , {Z}_{i}^{M} ) = \frac{{Z}_{i}}{α {Z}_{i}^{M}}$$

for some appropriate factor, α, used to condition the raw data, which conditioned data may be used to compute the diagnostic.

In a human subject, some body parts have homology in the body. For example, in females, the right breast has a homolog, namely the left breast. In a preferred embodiment of the invention, [symbol] ƒfₖ [symbol] is averaged over all the finite elements of the right breast to yield [symbol] ƒhd right [symbol] , and all the finite elements of the left breast to yield [symbol] ƒ_{left} [symbol] . In a different embodiment, [symbol] f_{right} [symbol] can refer to an average over finite elements belonging to a particular region within the right breast.

More generally, if the N finite elements comprising the grid are not all of equal size, the average is given by

$$⟨ {f}_{right} ⟩ = {\sum }_{k = 1}^{N} {p}_{k} ⟨ {f}_{k} ⟩ ,$$

where the probabilities pₖ are given by

Pₖ=χ_{A}(k)Vₖ/V_{A}.

In this last expression, _{χA}(k) is the characteristic function for a region A of the body part:

$${χ}_{A} ( k ) = \{ \begin{matrix}1 , & if finite element k ⋐ A \\ 0 , & otherwise\end{matrix}$$

and Vₖ and V_{A} are the volumes (if the grid is three dimensional) or the areas (if the grid is two-dimensional) of finite element k and region A, respectively.

The measured impedances in the body part are expected to be somewhat different from the values measured in the homologous body part. However, these differences are expected to be more pronounced if only one of these body parts contains a malignant tumor.

The comparator 26 includes hardware and/or software for comparing [symbol] ƒ_{left} [symbol] to [symbol] ƒ_{right} [symbol] to diagnose the possibility of disease. For example, if breast cancer is being diagnosed and if it is assumed that at least one breast is non-cancerous, then a difference between [symbol] ƒ_{left} [symbol] and [symbol] ƒ_{right} [symbol] may be due to a change in the electrical properties of one breast brought about by the presence of a cancer.

The comparator 26 calculates the absolute difference | [symbol] ƒ_{right} [symbol] − [symbol] f_{left} [symbol] |or a relative difference such as

$$( ⟨ {f}_{right} ⟩ - ⟨ {f}_{left} ⟩ ) / ⌊ \frac{1}{2} \cdot ( ⟨ {f}_{right} ⟩ + ⟨ {f}_{left} ⟩ ) ⌋$$

that is indicative of the possibility of disease in the body part or the homologous body part. Where there is a significant difference, further analysis can be performed to discern which of the homologous pairs may be cancerous. For example, as described above, it is known that the electrical properties of cancerous tissue deviate from the norm in a predictable way. Thus, the body part having electrical properties more like those of a cancerous body part can be suspect.

It should be understood that the principles of the present invention can be applied to diagnose disease in a body part without comparison to a homolog. For example, the diagnostic WEVal can be compared to a population average, to the baseline value, or to some other standard to diagnose disease.

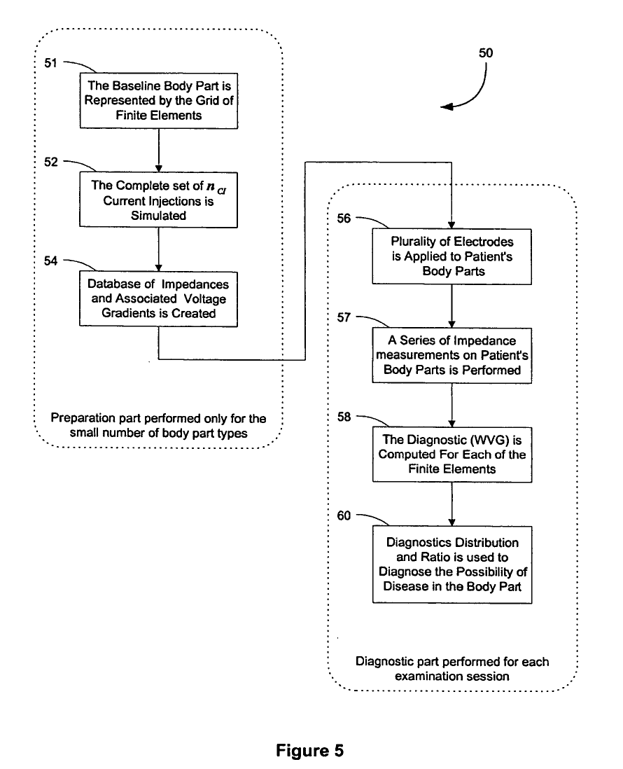

FIG. 5 shows a flowchart that illustrates the main steps 50 utilized by system 10 to diagnose the possibility of disease in a body part. The first part of the procedure is preparatory and establishes standard or idealized baselines for a typical body part and results are stored in the database to be used as a reference for numerous subjects. At step (51), the baseline body part is represented with a grid of finite elements. The grid can be two-dimensional, or three-dimensional. Next, at step (52), n_{CI} current injections are simulated to yield a database (54) of impedances and associated voltage gradients. These steps may be repeated to collect several typical sets of data depending on the size, body fat, or some other characteristic of the subject or the body part. This concludes the preparatory part. The subject-specific part of the procedure is described next. At step (56) a plurality of electrodes is applied to the body part, such as a breast and, at step (57), the plurality of electrodes measure impedance of the body part between electrode pairs. At step (58), a diagnostic is computed at each of the finite elements, the diagnostic being a function of the measured impedance and the values of impedance and gradients from the database. Subsequently, at step (60), the diagnostic is utilized to diagnose the possibility of disease in the body part.

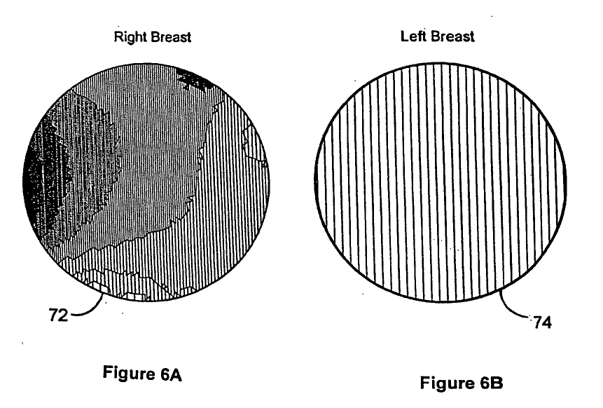

Referring to FIGS. 6A and 6B, sample results in the form of two gray scale plots are shown illustrating the value of the system and method of the present invention in diagnosing breast cancer. In FIGS. 6A and 6B, the right breast 72 and the left breast 74 are represented in the frontal plane as two circular plots, with darkness of gray increasing as the homologous difference of the diagnostic becomes more profound. This patient had an invasive ductal adenocarcinoma in the mid outer right breast. To generate these circular plots, each breast was represented by a circle with a 2D grid of finite elements. In FIGS. 6A and 6B, the finite elements comprising the grid are not shown.

The quantity | [symbol] ƒ_{right} [symbol] − [symbol] ƒ_{left} [symbol] | as calculated by the comparator 26 for homologous elements is, by convention, plotted on the side having the larger WEVal; i.e., on the right breast for elements where [symbol] ƒ_{right} [symbol] > [symbol] ƒ_{right} [symbol] (FIG. 6A) and on the left breast where [symbol] ƒ_{left} [symbol] > [symbol] ƒ_{right} [symbol] (FIG. 6B). These differences are scaled in the figure to the maximum level of black. Sixteen different levels of gray are presented, and some contrasting has been added to emphasize areas where the differences are highest. However, none of these scaling methods appreciably influenced the results. As can be seen in FIG. 6B, the shading in the normal left breast 74 is uniform (the light-most shade), indicating that for this subject [symbol] ƒ_{right} [symbol] > [symbol] ƒ_{left} [symbol] everywhere.

Different computer systems can be used to implement the method for diagnosing disease in a body part. The computer system can include a monitor for displaying diagnostic information using one of several visual methods. In one embodiment, the method can be implemented on a 2 GHz Pentium™ 4 system with 512 MB RAM.

It should be understood that various modifications and adaptations could be made to the embodiments described and illustrated herein, without departing from the present invention, the scope of which is defined in the appended claims. For example, although emphasis has been placed on describing a system for diagnosing breast cancer, the principles of the present invention can also be advantageously applied to other diseases of other body parts. These body parts need not have a homolog. Also, although the main measured electrical property described herein is impedance, it should be understood that other electrical properties, such as functions of the electrical impedance, may also be used in accordance with the principles of the present invention.

What is claimed is:

1. A method for diagnosing the possibility of disease in a body part, the method comprising
representing the body part with a grid having a plurality of finite elements;
obtaining a set of weights associated with a particular one of the plurality of finite elements using a model of the body part;
computing a diagnostic at the particular finite element, the diagnostic being a function of the set of weights, and a measured electrical property obtained with an electrode array; and
utilizing the diagnostic to diagnose the possibility of disease in the body part.

2. The method of claim 1, further comprising obtaining a baseline electrical property associated with the body part using the model thereof, wherein the diagnostic is a function of the baseline electrical property, the set of weights, and the measured electrical property obtained with the electrode array.

3. The system of claim 1, wherein the measured electrical property is conditioned to compute the diagnostic.

4. The method of claim 1, wherein the measured electrical property is an impedance.

5. The method of claim 1, wherein, in the step of representing, the grid is a two dimensional grid.

6. The method of claim 1, wherein, in the step of representing, the grid is a three dimensional grid.

7. The method of claim 2, wherein the baseline electrical property is obtained using a physical model of the body part.

8. The method of claim 2, wherein the baseline electrical property is obtained using a control subject.

9. The method of claim 2, wherein the baseline electrical property is obtained using a finite element method.

10. The method of claim 9, wherein the baseline electrical property is obtained by
obtaining a baseline voltage; and
using the baseline voltage to compute a baseline impedance.

11. The method of claim 10, wherein, in the step of obtaining a baseline electrical property, the model of the body part assumes a non-uniform resistivity.

12. The method of claim 1, further comprising
applying a plurality of electrodes to the body part; and
obtaining a measured electrical property of the body part with the plurality of electrodes.

13. The method of claim 12, wherein the step of applying includes
applying n_{CI} current injection electrode pairs on the body part, where n_{CI} is an integer greater than zero; and
applying n_{CI} voltage measurement electrode pairs on the body part, each of the current injection electrode pairs associated with one of the n_{CI} voltage measurement electrode pairs.

14. The method of claim 13, wherein the step of obtaining a measured electrical property includes
injecting a first current between a first pair of the n_{CI} current injection electrode pairs;
measuring the resultant voltage difference V₁^{M} between the voltage measurement electrode pair associated with the first current injection electrode pair;
repeating the preceding two steps of injecting and measuring with the other electrode pairs until all n_{CI} voltage differences, {V₁^{M}, V₂^{M}, . . . , Vₙ_{_{CI}}^{M}} are obtained; and
using the n_{CI} voltage differences to obtain associated measured impedances, {Z₁^{M}, Z₂^{M}, . . . , Zₙ_{_{CI}}^{M}}, where Zⱼ^{M} is the measured impedance obtained by using the j^{th} current injection electrode pair and the voltage measurement electrode pair associated therewith.

15. The method of claim 14, wherein, if the particular finite element is identified as the k^{th} finite element and the set of weights is denoted by {w₁ₖ,w₂ₖ, . . . , wₙ_{_{CI}}^{k}} where wᵢₖ is the weight associated with the k^{th} finite element and i^{th} current injection electrode pair, then the step of obtaining a set of weights, includes
using the model of the body part to obtain a set of current densities, {Jᵢₖ, J₂ₖ, . . . , Jₙ_{_{CI}}^{k}}, where Jᵢₖ is the current density at the k^{th} finite element when current is injected between the i^{th} current injection electrode pair; and
obtaining the set of weights using the relation
$${w}_{ik} = \frac{{J}_{ik}}{{\sum }_{j = 1}^{{n}_{CI}} {J}_{jk}} .$$

16. The method of claim 15, wherein the step of obtaining a baseline electrical property includes
using the model of the body part to obtain a set of baseline impedances {Z₁, Z₂, . . . , Zₙ_{_{CI}}} where Zᵢ is the impedance associated with the i^{th} electrode pair.

17. The method of claim 16, wherein the step of computing a diagnostic includes
calculating an average of a function ƒ(Zᵢ,Zᵢ^{M}) at the k^{th} finite element, the average given by
$$⟨ {f}_{k} ⟩ = {\sum }_{i = 1}^{{n}_{CI}} {w}_{ik} f ( {Z}_{i} , {Z}_{i}^{M} ) ,$$
wherein the diagnostic at the k^{th} finite element is defined to be (ƒₖ).

18. The method of claim 17, wherein the function ƒ(Zᵢ,Zᵢ^{M}) is given by
$$f ( {Z}_{i} , {Z}_{i}^{M} ) = \frac{{Z}_{i}}{{Z}_{i}^{M}} .$$

19. The method of claim 17, further comprising
obtaining diagnostics at each of the other finite elements, wherein the step of utilizing the diagnostic includes
averaging the diagnostics at each of the finite elements to find an averaged diagnostic [symbol] ƒ [symbol] ; and
calculating a second averaged diagnostic, [symbol] ƒₕₒₘₒ [symbol] , corresponding to a homologous body part.

20. The method of claim 19, wherein the step of utilizing the diagnostic further includes calculating a difference [symbol] ƒ [symbol] − [symbol] ƒₕₒₘₒ [symbol] wherein the quantity | [symbol] ƒ [symbol] − [symbol] ƒₕₒₘₒ [symbol] | is indicative of the possibility of disease in the body part or the homologous body part.

21. The method of claim 19, wherein the step of utilizing the diagnostic further includes calculating a quantity
$$\frac{⟨ f ⟩ - ⟨ {f}_{homo} ⟩}{\frac{1}{2} ( ⟨ f ⟩ + ⟨ {f}_{homo} ⟩ )}$$
that is indicative of the possibility of disease in the body part or the homologous body part.

22. A system for diagnosing the possibility of disease in a body part, the system comprising
a grid module for representing the body part with a grid having a plurality of finite elements;
a weight module for using a model of the body part to compute a set of weights associated with a particular one of the plurality of finite elements; and
a diagnostic module for computing a diagnostic at the particular finite element to diagnose the possibility of disease in the body part, wherein the diagnostic is a function of the set of weights, and a measured electrical property of the body part obtained with an electrode array.

23. The system of claim 22, wherein the grid module also obtains a baseline electrical property associated with the body part using the model thereof, the diagnostic being a function of the baseline electrical property, the set of weights, and the measured electrical property of the body part obtained with the electrode array.

24. The system of claim 22, wherein the grid module also conditions the measured electrical property to compute the diagnostic.

25. The system of claim 22, wherein the measured electrical property is an impedance.

26. The system of claim 22, wherein the grid is two dimensional.

27. The system of claim 22, wherein the grid is three dimensional.

28. The system of claim 22, wherein the model of the body part is a physical model.

29. The system of claim 28, wherein the physical model of the body part is associated with a control subject.

30. The system of claim 22, wherein the model of the body part is a numerical model that can be analyzed using a finite element method.

31. The system of claim 30, wherein the numerical model assumes a non-uniform resistivity.

32. The system of claim 22, further comprising an electrode array for obtaining the measured electrical property of the body part.

33. The system of claim 32, wherein the electrode array includes
n_{CI} current injection electrode pairs to apply on the body part, where n_{CI} is an integer greater than zero; and
n_{CI} voltage measurement electrode pairs to apply on the body part, each of the current injection electrode pairs associated with one of the n_{CI} voltage measurement electrode pairs.

34. The system of claim 33, wherein a first pair of the n_{CI} current injection electrode pairs transmits a first current through the body part;
the voltage measurement electrode pair associated with the first current injection electrode pair measures the resultant voltage difference V₁^{M}; and
the other electrode pairs inject and measure to obtain all n_{CI} voltage differences, {V₁^{M}, V₂^{M}, . . . , Vₙ_{_{CI}}^{M}}.

35. The system of claim 34, further comprising an impedance measuring instrument for measuring a set of impedance measurements {Z₁^{M}, Z₂^{M}, . . . , Zₙ_{_{CI}}^{M}} using the n_{CI} voltage differences, Z₁^{M} being the measured impedance associated with the i^{th} voltage electrode pair.

36. The system of claim 35, wherein the grid module includes
a finite element analysis module, which employs conditions corresponding to the injections of the currents between the pairs of current injection electrodes, to calculate an electrical potential as a function of position on the grid; and
a gradient module for using the electrical potential near the k^{th} finite element to compute a set of current densities, {J₁ₖ, J₂ₖ, . . . , Jₙ_{_{CI}}^{k}} where Jᵢₖ is the current density at the k^{th} finite element when current is injected between the i^{th} current injection electrode pair, wherein the set of weights are calculated according to
$${w}_{ik} = \frac{{J}_{ik}}{{\sum }_{j = 1}^{{n}_{CI}} {J}_{jk}}$$

37. The system of claim 36, wherein the grid module uses the model of the body part to obtain a set of baseline impedances
{Z₁, Z₂, . . . , Zₙ_{_{CI}}} where Zᵢ is the impedance associated with the i^{th} electrode pair.

38. The system of claim 37, further comprising
an averaging module for calculating an average of a function ƒ(Zᵢ,Zᵢ^{M}) at the k^{th} finite element, the average given by
$$⟨ {f}_{k} ⟩ = {\sum }_{i = 1}^{{n}_{CI}} {w}_{ik} f ( {Z}_{i} , {Z}_{i}^{M} ) ,$$
wherein the diagnostic at the k^{th} finite element is defined to be [symbol] ƒₖ [symbol] .

39. The system of claim 38, wherein the function ƒ(Zᵢ,Zᵢ^{M}) is given by
$$f ( {Z}_{i} , {Z}_{i}^{M} ) = \frac{{Z}_{i}}{{Z}_{i}^{M}} .$$

40. The system of claim 39, wherein
the electrode array, the grid module and the weight module are used to calculate diagnostics at the other finite elements, which together with the particular one, comprise the plurality of finite elements; and
the diagnostic module averages the diagnostics at the finite elements to find an averaged diagnostic [symbol] ƒ [symbol] , and calculates a second averaged diagnostic, [symbol] ƒₕₒₘₒ [symbol] , corresponding to a homologous body part.

41. The system of claim 40, wherein the diagnostic module calculates a difference [symbol] ƒ [symbol] − [symbol] ƒₕₒₘₒ [symbol] that is indicative of the possibility of disease in the body part or the homologous body part.

42. The system of claim 40, wherein the diagnostic module calculates a quantity
$$\frac{⟨ f ⟩ - ⟨ {f}_{homo} ⟩}{\frac{1}{2} ( ⟨ f ⟩ + ⟨ {f}_{homo} ⟩ )}$$
that is indicative of the possibility of disease in the body part or the homologous body part.
